(19) United States
(12) Patent Application Publication
(10) Pub. No.: US 2026/0165718 A1
(43) Pub. Date: Jun. 18, 2026

(54) THROMBUS REMOVAL SYSTEMS AND METHODS FOR BLOOD RETURN AND RECONSTITUTING REMOVED BLOOD CLOTS

(71) Applicant: Shifamed Holdings, LLC, Campbell, CA (US)

(72) Inventors: Aadel AL-JADDA, San Carlos, CA (US); Praveen Krishna DALA, San Ramon, CA (US); Oscar WILLIAMS, Los Gatos, CA (US); Matthew T. MUNOZ, Santa Cruz, CA (US); Yeeun CHO, San Jose, CA (US); Muralidharan SRIVATHSA, Santa Clara, CA (US); Joseph Creagan TRAUTMAN, Sunnyvale, CA (US); Tom SAUL, Portland, OR (US); Nicholas LYFORD, Saratoga, CA (US); Raymond HA, Milpitas, CA (US)

(21) Appl. No.: 19/123,732

(22) PCT Filed: Oct. 25, 2023
(86) PCT No.: PCT/US2023/077799

§ 371 Date: Apr. 23, 2025

Related U.S. Application Data
(60) Provisional application No. 63/380,779, filed on Oct. 25, 2022.
(60) Provisional application No. 63/380,876, filed on Oct. 25, 2022.
(60) Provisional application No. 63/502,040, filed on May 12, 2023.

(51) Int. Cl. A61B 17/22; A61B 17/00; A61M 1/00

(52) U.S. Cl. CPC A61B 17/22; A61M 1/60; A61B 2017/00544; A61B 2017/00561; A61B 2017/22079; A61B 2217/005; A61B 2217/007; A61M 2202/0021; A61M 2202/0028; A61M 2202/0042; A61M 2202/005; A61M 2202/0413; A61M 2202/06; A61M 2205/7545

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus.

### PRIORITY CLAIM

This patent application claims priority to U.S. provisional patent application no. 63/380,779, titled “THROMBUS REMOVAL SYSTEMS AND ASSOCIATED METHODS,” and filed on Oct. 25, 2022; U.S. provisional patent application no. 63/380,876 , titled “CLOT CATCHER AND BLOOD RETURN SYSTEMS AND METHODS FOR THROMBUS REMOVAL DEVICE,” and filed on Oct. 25, 2022; and U.S. provisional patent application no. 63/502,040, titled “THROMBUS REMOVAL SYSTEMS AND METHODS FOR RECONSTITUTING REMOVED BLOOD CLOTS,” and filed on May 12, 2023, which are herein incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the tortuous vascular anatomy, may not be effective in removing thrombotic material, and/or may lack the ability to provide sensor data or other feedback to the clinician during the thrombectomy procedure. Existing thrombectomy devices operate based on simple aspiration which works sufficiently for certain clots but is largely ineffective for difficult, organized clots. Many patients presenting with deep vein thrombus (DVT) are left untreated as long as the risk of limb ischemia is low. In more urgent cases, they are treated with catheter-directed thrombolysis or lytic therapy to break up a clot over the course of many hours or days. More recently other tools like clot retrievers have been developed to treat DVT and pulmonary embolism (PE), but these tools are not being widely adopted because of their limited effectiveness and additional costs versus aspiration or the standard of case. Other recent developments focus on slicing or macerating the clot, but these mechanisms are designed to reduce the risk of the catheter clogging and do not address the problem of tough, large, organized clots. There remains the need for a device to address these and other problems with existing venous thrombectomy including, but not limited to, a fast, easy-to-use, and effective device for removing a variety of clot morphologies.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports and fluid streams of a thrombus removal system according to embodiments of the present technology.

FIGS. 4A-4C illustrate various embodiments of a thrombus removal system including a saline source, an aspiration system, and one or more controls for controlling irrigation and/or aspiration of the system.

FIG. 5 illustrates an embodiment of a thrombus removal system that reduces the loss of blood.

FIG. 6 illustrates another embodiment of a thrombus removal system that reduces the loss of blood.

FIG. 7A illustrates a thrombus removal system with a thrombus filter and blood collection bags.

FIG. 7B illustrates a thrombus removal system with a thrombus filter, a vacuum chamber, and blood collection bags.

FIG. 7C is a flowchart describing a method of determining if aspirated fluid is to be returned to a patient.

FIGS. 8A-8D illustrate a thrombus removal system with a thrombus filter and blood collection cannister.

FIGS. 8E-8G illustrate variations of filters or clot catchers for separating clots from blood after removal by the thrombus removal device.

FIGS. 9A-9D illustrates a thrombus removal system with a thrombus filter and blood collection cannister.

FIGS. 10A-10D show another embodiment of a blood collection cannister.

FIGS. 11A-11C illustrate a structure that can be used in a thrombus filter or in a blood collection cannister.

FIGS. 12A-12C show another embodiment of a blood collection cannister.

### SUMMARY OF THE DISCLOSURE

A thrombus removal is provided, comprising an elongate shaft comprising a working end, at least one fluid lumen in the elongate shaft, and two or more apertures disposed at or near the working end, the two or more apertures in fluid communication with the least one fluid lumen and configured to generate two or more fluid streams to mechanically fractionate a target thrombus.

A thrombus removal system is provided, comprising an elongate shaft; an aspiration lumen extending along the elongate shaft; a vacuum source fluidly coupled to the aspiration lumen; a thrombus filter disposed along the aspiration lumen; and a blood collection cannister disposed proximally from the thrombus filter along the aspiration lumen, the blood collection cannister including a moveable separator that divides the blood collection cannister into first and second chambers, wherein operation of the vacuum source causes clot removed from the patient to collect on the thrombus filter and blood removed from the patient to collect in the first chamber.

In some aspects, operation of the vacuum source further causes saline to flow into the second chamber and a vacuum cannister.

In some aspects, the filter has a pore size of up to 40 microns.

In one aspect, the thrombus filter is configured to allow blood to pass but not allow thrombus to pass.

In some aspects, the thrombus filter is positively charged.

In one aspect, the separator comprises a plunger.

In some aspects, the separator comprises a diaphragm.

In other aspects, the system includes at least one saline source fluidly coupled to the second chamber.

In some aspects, the at least one saline source is removable from the blood collection cannister.

In one aspect, the system includes a blood return line fluidly coupled to the first chamber.

In another aspect, the thrombus filter comprises a honeycomb structure.

In some aspects, the honeycomb structure comprises a plurality of openings interspersed between closed sections.

In some aspects the system includes an electrical system configured to apply a positive charge to the closed sections.

A thrombus removal system is provided, comprising: an elongate shaft; an aspiration lumen extending along the elongate shaft; a vacuum source fluidly coupled to the aspiration lumen; and a blood collection cannister coupled to the aspiration lumen, the blood collection cannister including a sieve pathway having openings sized and configured to allow blood to flow out of the sieve pathway into the blood collection cannister while containing thrombus material within the sieve pathway.

In some aspects, the sieve pathway has a pore size of up to 40 microns.

In another aspect, the sieve pathway is positively charged.

In some aspects, the system includes a blood return line fluidly coupled to the blood collection cannister.

In one aspect, the sieve pathway comprises a honeycomb structure.

In some aspects, the honeycomb structure comprises a plurality of openings interspersed between closed sections.

In some aspects, the system includes an electrical system configured to apply a positive charge to the closed sections.

In one aspect, the sieve pathway is a spiral.

In other aspects, the sieve pathway takes a tortuous path through the blood collection cannister.

A thrombus removal system is provided, comprising an elongate shaft; an aspiration lumen extending proximally from the elongate shaft to a vacuum source; and a blood collection cannister disposed between the aspiration lumen and the vacuum source, the blood collection cannister including a positively charged conveyor belt configured to attract thrombus material from fluid within the blood collection cannister and a scraper configured to remove the thrombus material from the conveyor belt.

In some aspects, the scraper comprises a vacuum nozzle.

A method is provided, comprising: removing thrombus material and blood from a patient; applying a positive charge to a thrombus separation device; attracting thrombus material to the thrombus separation device; and allowing blood to flow into a blood collection cannister.

In some aspects, the thrombus separation device comprises a thrombus filter.

In other aspects, the thrombus separation device comprises a conveyor belt.

In some aspects, the thrombus separation device comprises a sieve pathway.

A thrombus removal system is provided, comprising: an elongate shaft; an aspiration lumen extending proximally in the elongate shaft to a vacuum source; a fluid lumen extending distally in the shaft from a pressurized fluid source; a thrombus detector operable to detect a thrombus between the aspiration lumen and the vacuum source; and a controller operable to adjust a flow of fluid through the fluid lumen and/or the aspiration lumen when a thrombus is detected by the thrombus detector.

In some aspects, the system includes a funnel disposed at or near a distal end of the elongate shaft.

A thrombus removal system is provided, comprising: an elongate shaft; an aspiration lumen extending proximally from the funnel in the elongate shaft to a vacuum source; a fluid lumen extending distally in the shaft from a pressurized fluid source; a thrombus filter disposed between the vacuum source and the aspiration lumen; and one or more blood collection bags disposed between the thrombus filter and the vacuum source.

In some aspects, the system includes a funnel disposed at or near a distal end of the elongate shaft.

A method is provided, comprising: initiating a thrombectomy procedure in a patient with at thrombectomy device; identifying a system state of the thrombus removal device; determining if fluid aspirated by the thrombus removal device is to be returned to the patient or if the fluid is waste based on the system state; and directing the fluid into a selected receptacle.

In some aspects, a first system state is when aspiration of the thrombus removal device is turned on and jetting or fluid delivery of the thrombus removal device is turned off.

In another aspect, the method includes determining that the fluid aspirated by the thrombus removal device is to be returned to the patient in the first system state.

In some aspects, a second system state is when aspiration of the thrombus removal device is turned on and jetting or fluid delivery of the thrombus removal device is turned on.

In one aspect, the method includes determining that the fluid aspirated by the thrombus removal device is waste in the second system state.

In some aspects, directing the fluid into the selected receptacle comprises automatically controlling one or more valves to direct the fluid into the selected receptacle.

A thrombectomy method is also provided, comprising: engaging a clot with a thrombus removal device; directing two or more fluid streams into the clot with the thrombus removal device to macerate the clot; sorting macerated portions of the clot into a clot collection cannister of the thrombus removal device based on a parameter of the macerated portions.

In some aspects, the parameter comprises a size of the macerated portions.

In other aspects, the parameter comprises a morphology of the macerated portions.

In some aspects, the parameter comprises a hardness of the macerated portions.

In some aspects, the method includes indicating to a user a volume of the macerated portions.

In one aspect, indicating to the user comprises indicating the volume to the user with one or more measurement markers on the clot collection cannister.

In another aspect, the macerated portions are sorted with differential momentum.

In some aspects, sorting the macerated portions further comprises applying one or more electrical charges to elements within the clot collection cannister to attract selected macerated portions.

### DETAILED DESCRIPTION

This application is related to disclosure in International Application No. PCT/US 2021/020915, filed Mar. 4, 2021 (the '915 application), and International Application No. PCT/US 2022/033024, filed Jun. 10, 2022 (the '024 application), the disclosures of which are incorporated by reference herein for all purposes. The '915 and '024 applications describe general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

It is an object of this disclosure to provide thrombectomy systems and methods configured to separate clot from aspirated blood. This can include systems and methods for presentation/measurement of clot removed. In some implementations, the clot removed from a patient can be separated and presented visually to a user of the system, such as in a clot catcher, filter, or waste container of the system. In other examples, the clot can be presented on a screen (e.g., photos, videos, or digital representations of the clot).

It is another object of this disclosure to prepare blood for reuse and/or redelivery to the patient. This can include separating removed blood from clot (i.e., filtering) and automatically or manually injecting or delivering the blood back into the patient. Blood return can include systems and techniques that minimize contact with air, or alternatively replace air with CO₂, He, or other gases.

The systems and methods provided herein can include various techniques for separating clot from blood. This can include size exclusion/trapping (e.g., with traditional filters such as membrane), depth filtering (e.g., pores or tortious path which additionally can take advantage of viscosity of blood relative to clot), delta flow, size separation such as flow in velocity field gradients (e.g., using a cyclone filter, inertial particle motion, Couette flow rotating plates or filters), or affinity (e.g., charge, antibody, or collision).

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance.

Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to render the structure and/or consistency of the clot such that it is more easily transported through the aspiration system (e.g., fragment the thrombus with pressurized fluid), an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, in addition to or alternatively to high pressure fluid the aspiration pump may provide lower pressure fluid within the aspiration lumen near the proximal portion of the thrombus removal system, urging aspiration of thrombus from the distal portion.

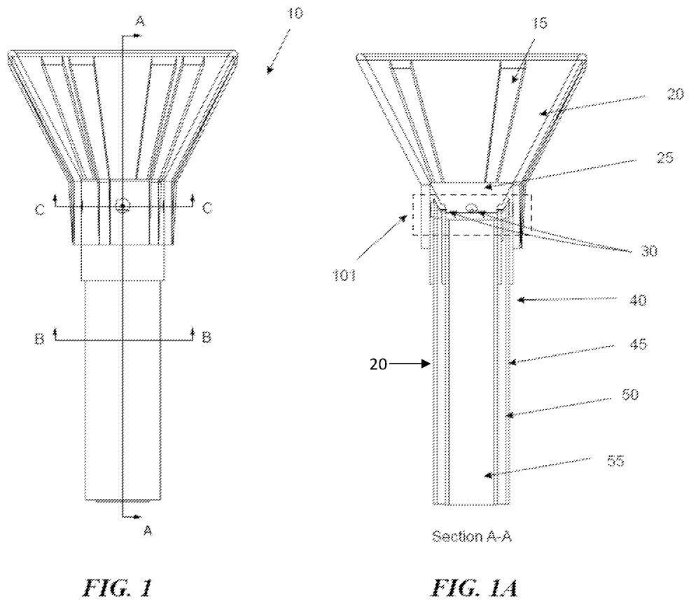

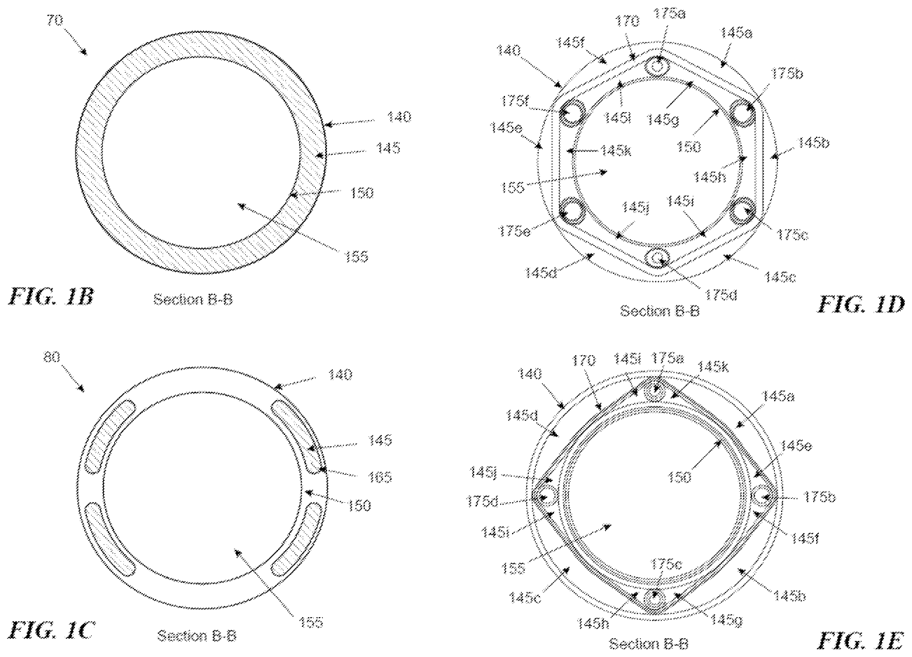

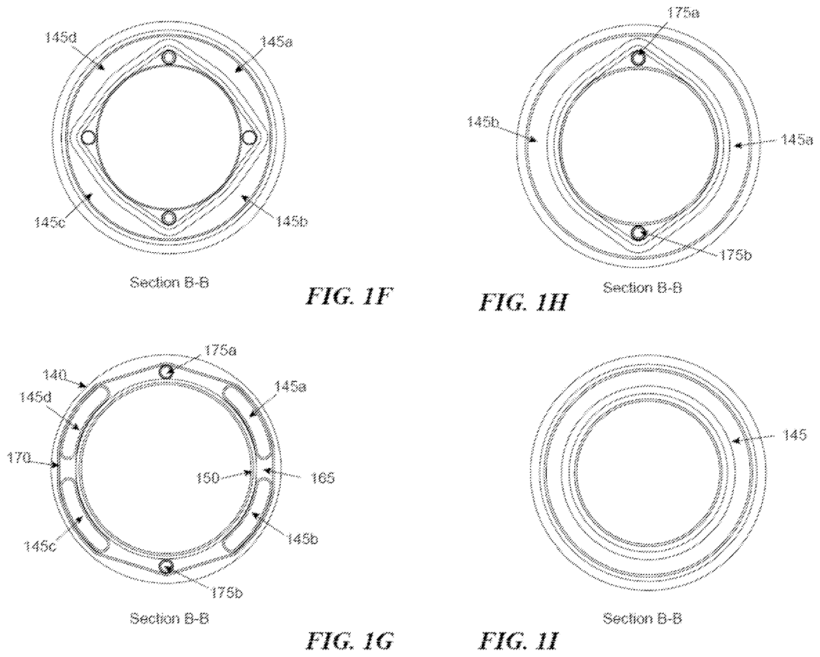

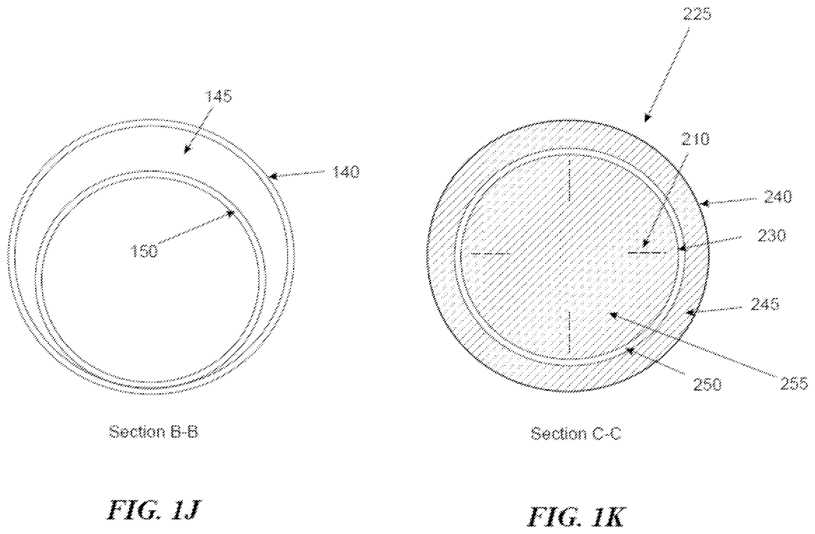

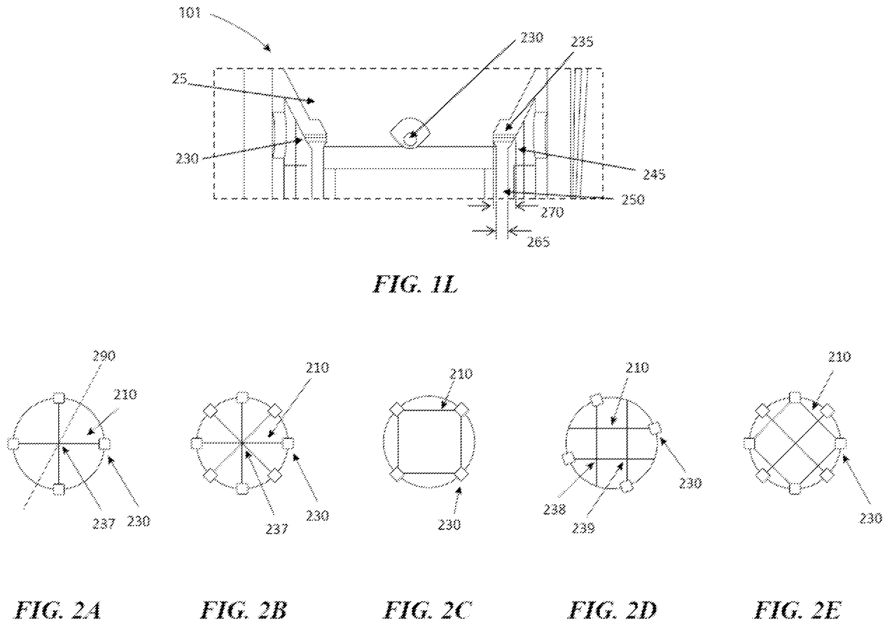

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus within a blood vessel and/or a tissue (e.g., vessel) wall to aid in thrombus fragmentation and/or removal. The funnel can have a variety of shapes and constructions as would be understood by one of skill from the description herein. The thrombus removal system may be delivered through a sheath to a thrombus site in a blood vessel with funnel 20 in a compressed configuration. Funnel 20 may self-expand as it is advanced out of the sheath and/or as the sheath is retracted from the funnel.

The example section A-A in FIG. 1A depicts a double walled thrombus removal device construction having a catheter 22 extending proximally from funnel 20 with an outer wall/tube 40 and an inner wall/tube 50. An aspiration lumen 55 is formed by the inner wall 50 and is centrally located. Aspiration lumen 55 communicates with a vacuum source, as described below. A generally annular volume forms at least one fluid lumen 45 between the outer wall 40 and the inner wall 50. The fluid lumen 45 is adapted for fluid communication with a fluid delivery mechanism, as described below. One or more apertures (e.g., nozzles, orifices, or ports) 30 are positioned in the thrombus removal system to be in fluid communication with the fluid lumen 45 and an irrigation manifold 25 at the base of or within funnel 20. In operation, the ports 30 are adapted to direct (e.g., pressurized) fluid toward thrombus material that is engaged with the distal portion 10 of the thrombus removal system to macerate, fragment, or cut the thrombus material. Aspiration lumen 55 pulls thrombus material along with fluid from ports 30 and blood from the blood vessel proximally to a receptacle outside of the patient, as described below.

In various embodiments, the system can have an average flow velocity within the fluid lumen of up to 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be up to 20 m/s, the peak fluid velocity in the lumen may be up to 30 m/s or more during the pulsing of the fluid source. In some embodiments, the jets or apertures are no smaller than 0.0100″ or even as small as 0.008″ to avoid undesirable spraying of fluid. In some embodiments, the system can have a minimum aspiration pressure of 1 or 2 inHg absolute, to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally similar features and components described herein may be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-141 and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The sensors can be used during therapy to provide feedback for the system (e.g., sensors can be used to detect clogs to initiate a clog removal protocol, or to determine the proper therapy mode based on sensor feedback such as jet pulse sequences, aspiration sequences, etc.). The auxiliary ports can therefore be used to connect to the sensors, e.g., by electrical connection, optical connection, mechanical/wire connection, and/or fluid connection. It is also contemplated that the fluid and auxiliary lumens can be configured to carry and deliver other fluids, such as thrombolytics or radio-opaque contrast injections to the target tissue site during treatment.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order.

In various embodiments, the fluid pressure is generated at the pump (in the console or handle). The fluid is accelerated as it exits the ports at the distal end and is directed to the target clot. In this way a wider variety of cost-effective components can be used to form the catheter while still maintaining a highly-effective device for clot removal. Additional details are provided below.

FIG. 1E, Section B-B, illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145e-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment 1F includes the same four auxiliary ports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the exemplary outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plan view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 at the base of the funnel that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values.

The manifold is configured to increase a fluid pressure and/or flow rate of the fluid. When fluid is provided by the fluid delivery mechanism to the fluid lumen(s) at a first pressure and/or a first flow rate, the manifold is configured to increase the pressure of the fluid to a second pressure and/or is configured to increase the flow rate of the fluid to a second flow rate. The second pressure and/or second fluid rate can be higher than the first pressure and/or first flow rate. As a result, the manifold can be configured to increase the relatively low operating pressures and/or flow rates generated by the fluid delivery mechanism to the relatively high pressures and/or high flow rates generated by the ports/fluid streams.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which multiply with respect to individual fluid streams that are not directed to combine at the intersection. The increased fluid momentum and/or energy transfer at an intersection may advantageously fragment thrombus more efficiently and/or quickly. As described above, in some embodiments, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15 m/s to up to 150 m/s. At these higher velocities (e.g., above 15 m/s, or alternatively above 20 m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams should be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e., speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

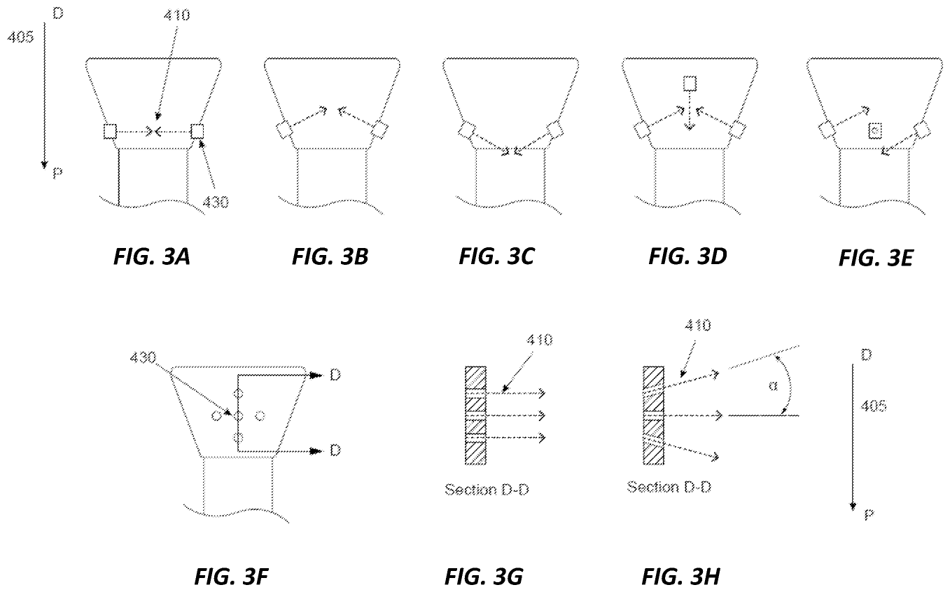

FIGS. 3A-3H depict various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle a may characterize an angle that a fluid stream 410 is directed with respect to an axis that is orthogonal to the flow axis 405 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

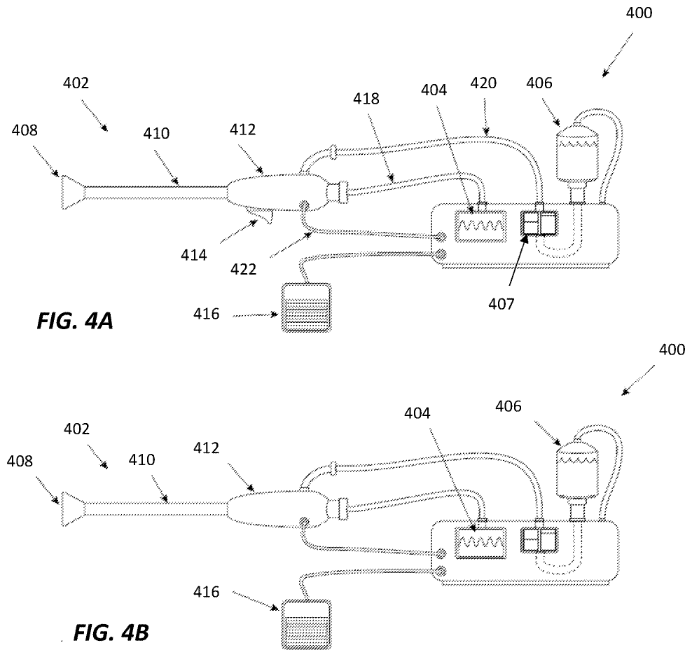

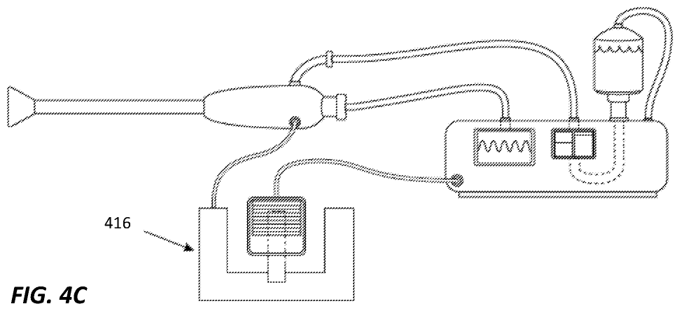

FIGS. 4A-4C illustrate various configurations of a thrombus removal system 400, including a thrombus removal device, 402, a vacuum source and cannister 404, a fluid source 406, and a pump 407. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 402 can include a funnel 408, a flexible shaft 410, a handle 412, and one or more controls 414 and 416. For example, in the embodiment shown in FIG. 4A, the device can include a finger switch or trigger 414 and a foot pedal or switch 416. These can be used to control aspiration and irrigation, respectively. Alternatively, as shown in the embodiment of FIG. 4B, the device can include only a foot switch 416, which can be used to control both functions, or in FIG. 4C, the device can include only an overpedal 416, also used to control both functions. It is also contemplated that an embodiment could include only a finger switch to control both aspiration and irrigation functions. As shown in FIG. 4A, the vacuum source and cannister 404 can be coupled to the aspiration lumen of the device with a vacuum line 418. Any clots or other debris removed from a patient during therapy can be received by, and stored in, the vacuum cannister 404 for later disposal. Similarly, the fluid source 406 (e.g., a saline bag) can be coupled to the fluid lumens of the device with a fluid line 420 for delivery of high-pressure and velocity fluid streams or jets at the base of funnel 408 to fragment thrombus material engaged by funnel 408, as described above.

Still referring to FIG. 4A, electronics line 422 can couple any electronics/sensors, etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both systems in response to sensor data, such as pressure data, flow data, etc.

Referring to FIGS. 1A and 4A-4C, prior to introduction of the thrombus removal device into a patient's blood vessel, the system is primed to remove air by pumping fluid through fluid lumen 45, manifold 25, ports 30, aspiration lumen 55, and vacuum line 418. During a thrombus removal procedure, the thrombus removal device is advanced into the patient's blood vessel, and the funnel is expanded to engage the thrombus. Pressurized fluid is delivered from fluid source 406 and pump 407 through fluid line 420 and fluid lumen 45 to manifold 25 and ports 30. Simultaneously, vacuum is applied to aspiration lumen 55 by vacuum source and cannister 404. When a clot is engaged, the material flowing proximally through aspiration lumen 55 to the vacuum source and cannister is primarily fluid delivered through ports 30 combined with any blood that is able to pass around the engaged thrombus. As the fluid jets begin to break up the thrombus, thrombus material is pulled proximally through aspiration lumen 55 along with the injected fluid and any blood that can pass into the funnel around the thrombus. After the thrombus has been broken up sufficiently to become dislodged from the blood vessel, the remaining thrombus material moves proximally toward the vacuum source and cannister 404.

In some embodiments, the controller can reduce the strength of the vacuum applied to the aspiration lumen after a thrombus has been detected in the vacuum line so that blood loss is minimized.

As is described above, aspiration occurs down the central lumen of the device and is provided by a vacuum pump in the console. The vacuum pump can include a container that collects any thrombus or debris removed from the patient.

When the thrombus has been broken up and removed, blood flow in the blood vessel will be restored, and any further application of vacuum will aspirate the patient's blood along with fluid supplied through ports 30. After removing a thrombus, it may be desirable to determine or visualize the volume of removed thrombus and/or capture and return blood to the patient to minimize loss without returning any portions of the thrombus.

A sensor may be used to detect movement of the thrombus into cannister 404 so that aspiration of the patient's blood can be minimized. For example, a camera may be used to detect passage of the thrombus through aspiration lumen 55 (e.g., as aspiration lumen 55 passes through catheter handle 412), through vacuum line 418, or in vacuum source and cannister 404.

Alternatively, because the pressure in aspiration lumen 55 and vacuum line 418 will increase when the thrombus is dislodged and drawn proximally, a pressure sensor communicating with aspiration lumen 55 and/or vacuum line 418 may detect removal of the thrombus. Additional details on using sensing to detect removal of a thrombus can be found in International App. No. PCT/US2022/033024, filed Jun. 10, 2022, which is incorporated herein by reference.

The pressure sensor may be disposed, e.g., within the handle of the catheter (so that it communicates with the portion of aspiration lumen just distal to catheter 20) or at the cannister of the vacuum source. The system may then reduce or stop fluid supplied to ports 30 of funnel 408 and aspiration back through funnel 408 into aspiration lumen 55 to reduce the aspiration of blood, e.g., by turning off vacuum source and cannister 404 and pump 407.

The system may detect a removed thrombus before the thrombus reaches the vacuum cannister 404. While aspiration must still be applied to move the thrombus proximally the remaining distance to the vacuum cannister (now shown), any aspiration applied to the funnel within the blood vessel will continue to draw in the patient's blood.

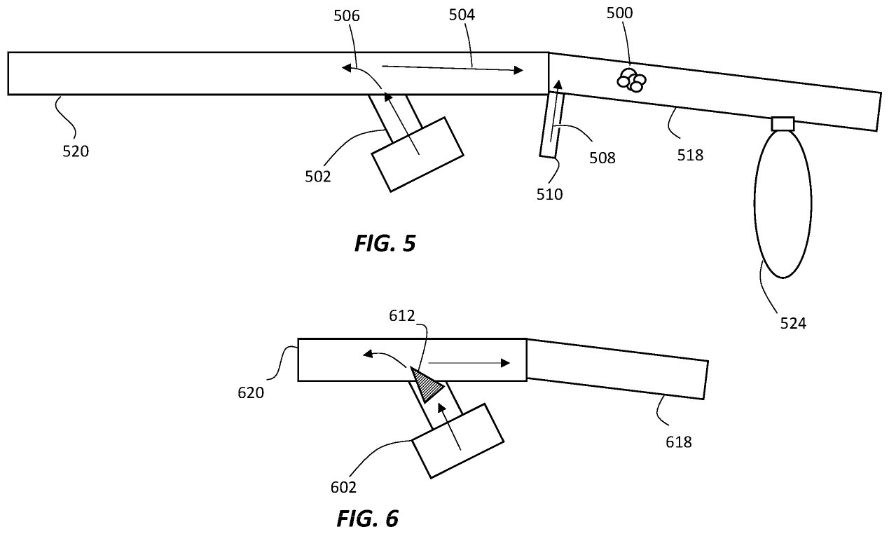

As an alternative, as shown in FIG. 5, when a thrombus 500 is detected in vacuum line 518, additional fluid from the fluid source and pump (not shown, but can be, for example, fluid source 406 in FIG. 4A) can be supplied to catheter 520 distal to vacuum line 518 through a Y junction 502. A portion of the additional fluid will be drawn proximally into vacuum line 518 (as shown by arrow 504) to help move the thrombus further proximally to the vacuum cannister 524. Any additional fluid added in this manner will reduce the amount of blood aspirated from the patient. An additional fluid flow rate that equals the aspiration flow rate will stop the removal of the patient's blood. If the flow rate of the additional fluid is greater than the aspiration rate, a portion of the additional fluid (as shown by arrow 506) will flow distally through the aspiration lumen of catheter 520 to return any blood within the aspiration lumen to the patient. Additionally or alternatively, additional fluid from the fluid source and pump may be added through a port 510 into a distal portion of vacuum line 518 (as shown by arrow 508) to help move the thrombus further proximally in the vacuum line while also reducing the amount of fluid (such as the patient's blood) being pulled proximally through the aspiration lumen. The funnel at the distal end of catheter 520 may be re-sheathed after the thrombus has been detected in aspiration line 518 to reduce the flow of blood proximally into catheter 520.

FIG. 6 is an alternative embodiment of the system of FIG. 5, which shows an optional valve 612 (e.g., a flap valve) in Y junction 602 that may be operated to control the ratio of fluid flowing distally into catheter 620 to the amount of fluid flowing proximally into vacuum line 618.

When the thrombus has been broken up and removed, blood flow in the blood vessel will be restored, and any further application of vacuum will aspirate the patient's blood along with fluid supplied through ports 30. After removing a thrombus, it may be desirable to capture and return blood to the patient to minimize loss without returning any portions of the thrombus.

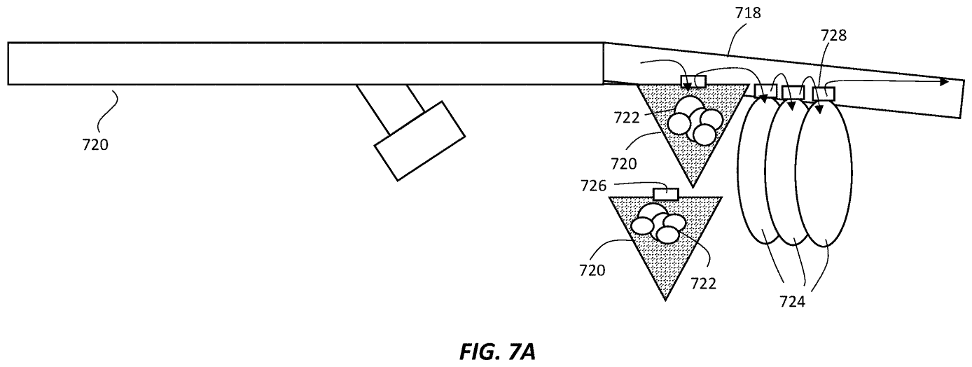

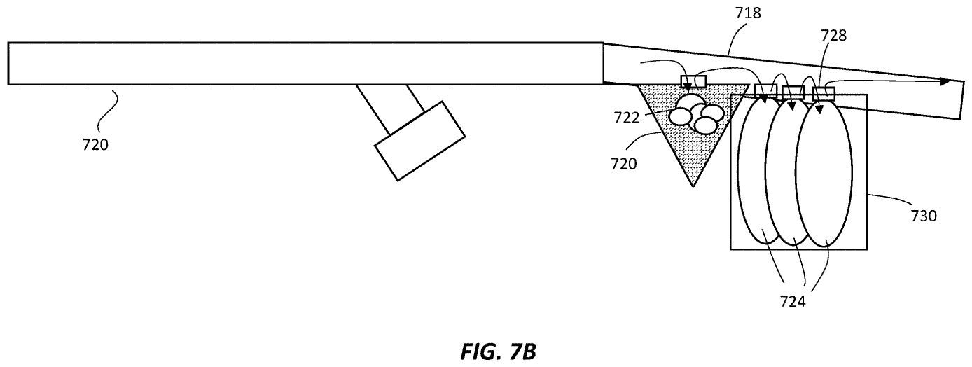

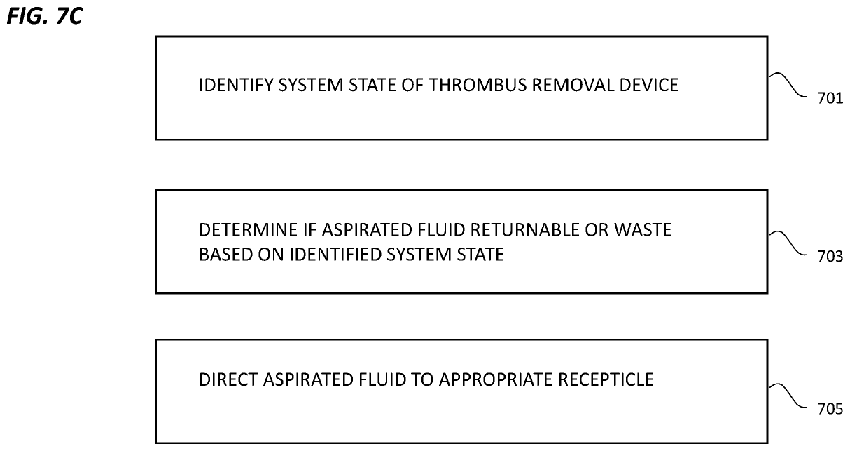

FIG. 7A shows features enabling the separation of thrombus material from removed blood and the capture of the filtered blood for return to the patient. Proximal flow of the aspiration fluid in vacuum line 718 passes through a thrombus filter 720 that removes thrombus material 722 from the flowing fluid (i.e., thrombus, blood, and aspirated fluid from the fluid jets). The filter 720 may be detached and replaced using, e.g., a coupler 726 as it fills with thrombus material.

After leaving the filter 720, the filtered fluid (e.g., blood) returns to vacuum line 718 and flows proximally into one or more sterile, collapsed collection bags 724 arranged serially along line 718. Dividers within the bags 724 or within their couplers 728 can direct flowing fluid into and out of the bags. As each bag 724 fills, the filtered fluid begins to fill the next bag. The collection bags 724 may be removed via couplers 728 so that the collected blood can be returned to the patient.

In some aspects, the couplers 728 can comprise controllable valves (e.g., electronically actuated valves) to selectively control which of the collection bags are open to receive flowing fluid from the vacuum line. Control of the valves can be based on, for example, a system state of the thrombus removal device (e.g., aspiration on/off and/or jetting on/off, or any combination thereof). For example, during periods of operation in which only aspiration is active and jetting is turned off, only filtered blood will pass through filter 720 into the selected collection bags. However, if aspiration and jetting are both turned on, then the fluid flowing into the controlled bags will include a combination of jetting fluid (e.g., saline) and filtered blood. It may be desirable to avoid returning blood to the patient that has potentially been lysed with jetting/saline.

In some aspects, the control of valves within the couplers 728 can be according to a system state. The system state can be associated with the type of fluid that is being aspirated, and can also be associated with whether or not the fluid is to be returned to a patient. In one implementation, the thrombus removal device can include two distinct system states for the purposes of blood collection/return: 1) Aspiration on; jetting/fluid delivery off, and 2) Aspiration on; jetting/fluid delivery on. In the first system state, since the jetting/fluid delivery is turned off, it can be assumed that all fluid collected in the aspiration lumen of the device comprises either blood or blood with removed/macerated thrombus. Once the thrombus is filtered out of the fluid, the fluid entering collection bags 724 can be assumed to be filtered blood suitable for return to a patient. The valves or couplers 728 of one or more of the collection bags 724 can be controlled to be opened to allow for this filtered blood to be collected for potential return to the patient. In the second system state described above, it can be assumed that the fluid passing through the filter is a combination of blood and jetting/irrigation fluid such as saline, with the clots being filtered out by filter 720. In some situations, a physician or the system may determine that it is acceptable to return the filtered blood/saline to the patient. In other situations, the physician or system may determine that the risk of lysing the blood is too great to return the blood/saline to the patient. In some aspects, the valves or couplers of one or more of the collection bags may be controlled to separate this blood/saline into a collection bag that is marked or tagged as containing a combination of blood and saline. In yet additional embodiments, one or more of the collection bags can be identified as a “blood return” bag and one or more of the collection bags can be identified as a “waste” bag. The couplers/valves can be automatically controlled based on the system state described above. If only aspiration is turned on and jetting is off, then the valves associated with the “blood return” bags can be opened and the valves associated with the “waste” bags can be closed. Likewise, if aspiration and jetting are both turned on, then the valves associated with the “blood return” bags can be opened and the valves associated with the “waste” bags can be closed.

FIG. 7C is a flowchart describing the method described above. At step 701 of the flowchart, the method can include identifying a system state of the thrombus removal device. As described above, the key operating parameters used to determine system state are if aspiration is turned on or off and if jetting/fluid delivery is turned on or off. In one implementation, two system states are considered: 1) Aspiration on; jetting/fluid delivery off, and 2) Aspiration on; jetting/fluid delivery on.

At step 703 of the flowchart, the method can include determining if the fluid aspirated by the thrombus removal device is returnable to the patient or if the fluid aspirated by the thrombus removal device is waste. This determination can be based on the identified system state from step 701. For example, in some implementations, aspirated fluid is safe to return to a patient if aspiration is on and jetting is off. In some embodiments, aspirated fluid is also safe to return if aspiration is on and jetting is on. However, in other embodiments, aspirated fluid is not safe to return if aspiration is on and jetting is on due to the risk of lysing the blood with the jetting/fluid delivery. In some embodiments, a user or physician can determine whether each system state is associated with fluid that is safe to return or with fluid that is waste.

At step 705, the method can further include directing the aspirated fluid to the appropriate receptacle of the thrombus removal device. This can be based on whether or not the fluid is safe to return or waste. In some embodiments, if the fluid is safe to return, it can be directed to a blood return container or receptacle (such as by controlling one or more valves) to divert the aspirated fluid into the blood return receptacle. In some embodiments, if the fluid is waste, it can be directed to a waste container or receptacle (such as by controlling one or more valves) to divert the aspirated fluid into the waste receptacle. In some embodiments, this valve control can be controlled automatically. In some aspects, the timing of controlling the valves can account for the volume of fluid that is contained between a distal tip of the thrombus removal device and the appropriate receptacle. For example, if jetting is suddenly turned on after aspirating with jetting off, the aspiration lumen will likely be full of blood that is returnable. The timing of the valves can be gated or synchronized to direct this returnable blood into the appropriate container, before switching the valves to direct the combination of blood/saline into, for example, a waste container.

Referring to FIG. 7B, another embodiment is provided similar to the embodiment of FIG. 7A, but in this embodiment the collection bags 724 are placed within a vacuum chamber 730. In some embodiments, the vacuum chamber 730 can be connected to the aspiration source of the thrombus removal device. In other embodiments, the vacuum chamber can have a separate vacuum source (not illustrated). In this example, vacuum is applied to the vacuum chamber which “inflates” the bag with blood as blood and clots are aspirated out of the patient.

In some embodiments, the vacuum chamber 730 can be detachable from the thrombus removal device and can come pre-loaded with the collection bags. When the bags are filled with blood, the vacuum chamber can be replaced with a new vacuum chamber with new unfilled collection bags. Alternatively, the collection bags can be engaged serially within the vacuum chamber and replaced individually when full.

In some embodiments, the controller can reduce the strength of the vacuum applied to the aspiration lumen after a thrombus has been detected in the vacuum line so that blood loss is minimized.

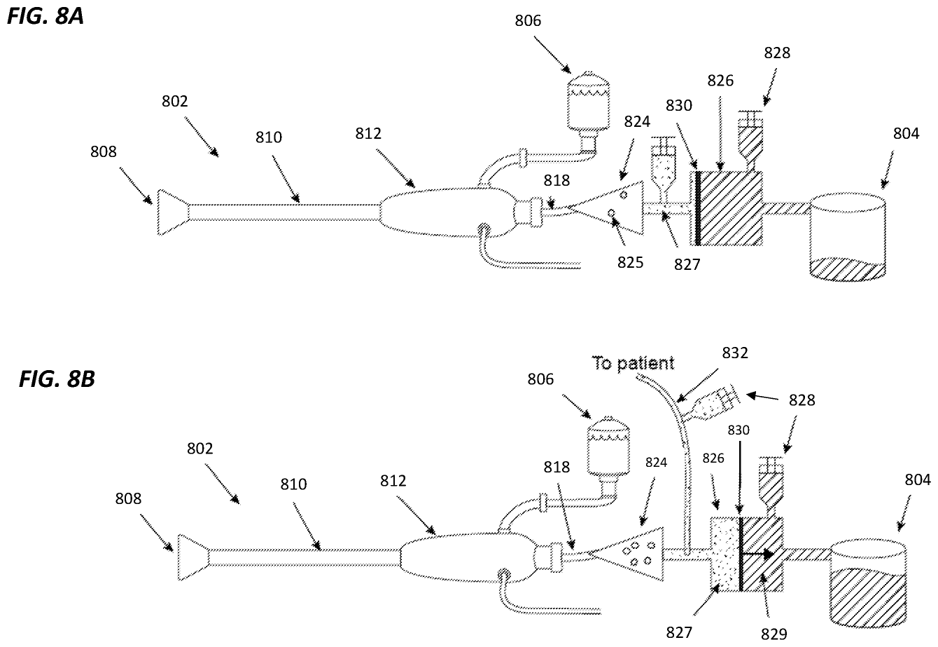

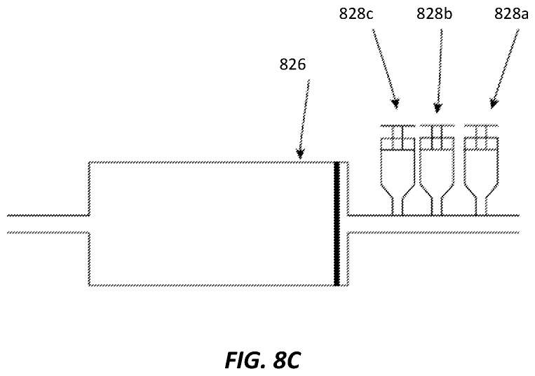

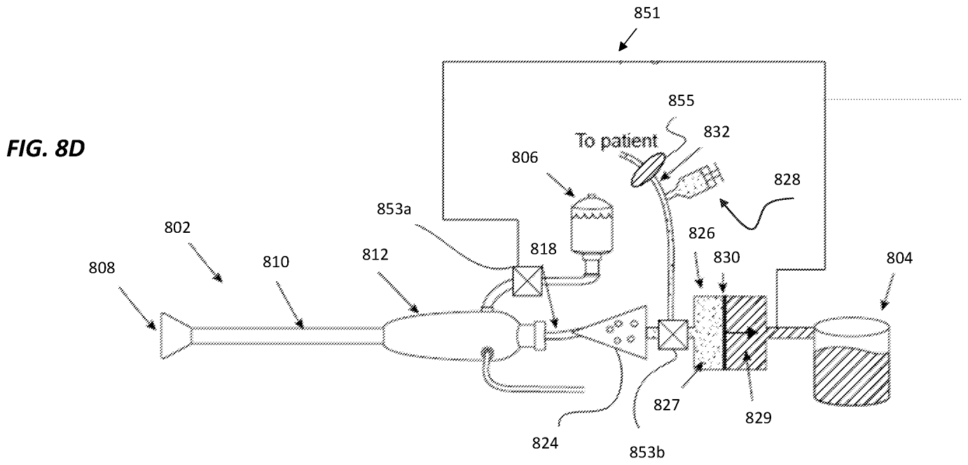

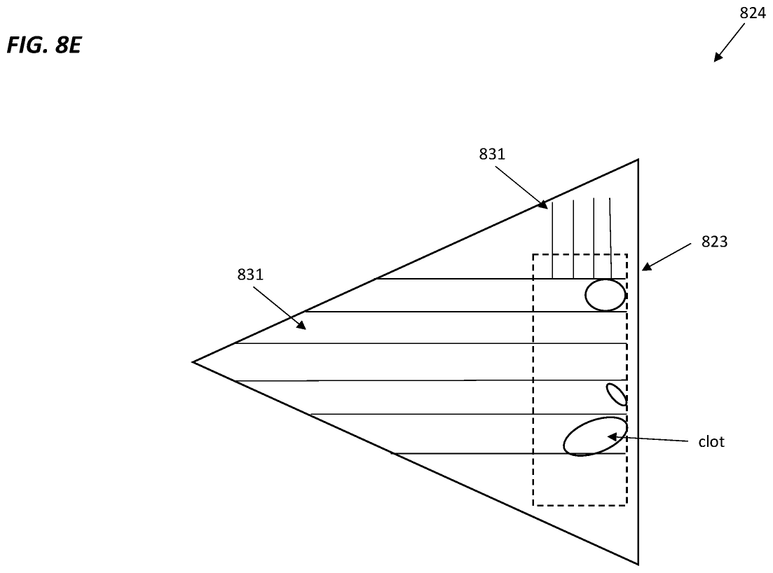

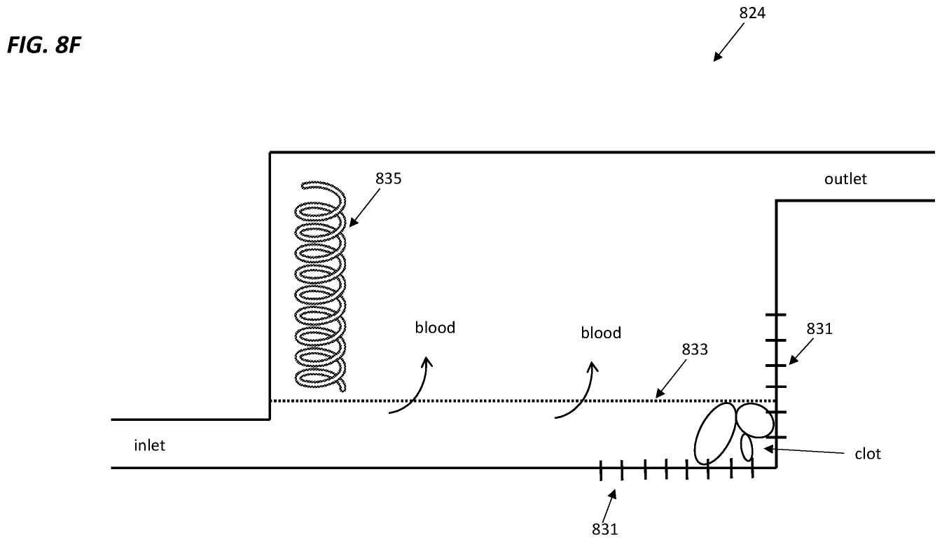

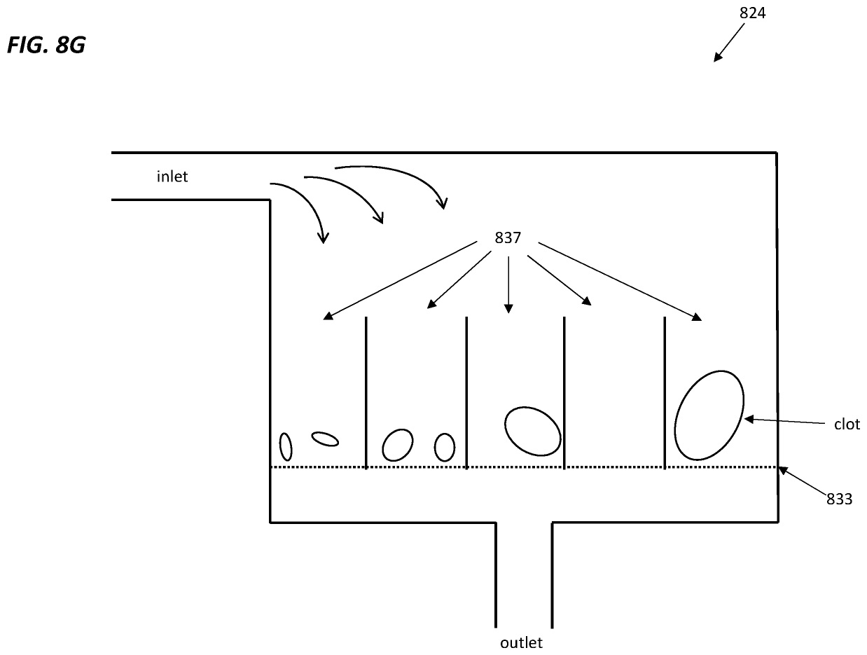

FIGS. 8A-8C, 9A-9B, 10A-10C, 11A-11C, and 12A-12B show features enabling the separation of thrombus material from removed or aspirated blood and the capture of the filtered blood for return to the patient. In some embodiments, the separated thrombus material can be easily viewed by the physician to provide a real-time indication on the amount/size/volume of thrombus material removed from the patient during a procedure.

Referring to FIGS. 8A-8B, a thrombus removal system can include previously described components including a thrombus removal device 802, a vacuum source and cannister 804, a fluid source 806, and one or more pumps (not shown). The thrombus removal system can further include a funnel 808, a flexible elongate shaft 810, and a handle 812. The thrombus removal system can further include a thrombus filter 824 and a blood collection cannister 826 fluidly coupled to the vacuum line 818. The blood collection cannister 826 can further include one or more syringes 828 (or other transfer device) and a separator 830 configured to fluidly separate the blood collection cannister into two separate chambers. The syringes can be optionally removable from the blood collection cannister. In some embodiments, the syringes are located on the blood side of the cannister, the saline side of the cannister, or both. For example, syringes on the blood side of the cannister can be used to return blood to the patient (e.g., by removing them from the cannister and injecting them back into the patient, or into a line connected to the patient. Syringes on the saline side can be used to optionally pull saline for injection into the patient or to increase/decrease a volume of saline in the blood collection cannister. The separator can comprise, for example, a plunger, a diaphragm, a fluid impermeable membrane, or the like. In general, the separator is configured to separate blood/saline removed from the patient from saline or other fluid inside the blood collection cannister during operation of the vacuum source.

It should be understood that many of the components described above can be incorporated into a separate console, such as the vacuum source and cannister, blood collection cannister, etc. In some embodiments, the thrombus removal device 802 is positioned within a sterile field, and the console and/or other components can be positioned outside of the sterile field. In some embodiments, however, it may be desirable to position certain components, such as the thrombus filter 824, within the sterile field so that a user of the device, such as a physician, can view the amount of removed thrombus easily in real-time during a procedure.

When the vacuum source is activated and thrombus is engaged at the funnel, blood and/or thrombus material flows from the funnel 808 into an aspiration lumen of the shaft 810 and into vacuum line 818, then through thrombus filter 824. In some embodiments the thrombus filter can be a simple size exclusion filter with an effective pore size configured to remove thrombus material 822 from the flowing fluid while allowing blood and/or saline to pass through the filter. For example, the filter may have a 40 micron (or smaller) pore size corresponding to many conventional or traditional filters for red blood cells. Red blood cells typically have a diameter ranging from 7.5 to 8.7 μm in diameter and 1.7 to 2.2 μm in thickness. Other appropriate pore sizes are within the scope of this disclosure that allow blood/saline to pass through the filter while not allowing or minimizing the passage of thrombus material. The separated thrombus material 825 can collect on the filter 824.

In some embodiments, the filter 824 can include clot-adherent materials (e.g., polyesters) configured to grab or adhere to passing clots. Portions of the filter may further include clot-repellant materials (e.g., ePTFE) to selectively allow clot to pass through certain areas or portions of the filter.

Referring to FIG. 8E, the thrombus filter 824 can be housed within a transparent container or can include a transparent window 823 and include one or more spring loaded channels 831 which causes the clot to be pressed against an inner surface of the container or transparent window to enable visualization of removed clots. In some aspects, the window or transparent housing can include markers 831 configured to indicate a unit of measurement of the captured clot to the user. For example, the markers 831 can be graduated or spaced apart by known units of volume (e.g., every 5 ml of removed clot) to give the user a quick estimate of the amount of removed clot during a procedure. In other embodiments, weigh scales can be used in the clot collection cannister to indicate a weight of the clot removed. In some aspects, the weight can be converted to an estimated volume of the clot removed. The filter 824 and blood collection cannister can be fully fluidized, which can prevent coagulation during a thrombectomy procedure.

In another embodiment, referring to FIG. 8F, a filter 824 can comprise a filter that uses gravity to separate clots from blood after removal by the thrombectomy device. As shown in FIG. 8F, blood and clot can enter the filter 824 through the inlet. A membrane or filter 833 can comprise a coarse filter that has a pore size that allows blood to flow through while preventing clot or thrombus material from passing. In some aspects, the filter or membrane can be weighted. In other embodiments, a weighted bar can be attached or coupled to the membrane to compact the collected clot at the bottom of the filter. In some embodiments, the filter 824 can optionally include a spring-loaded mechanism 835 that can apply force against the filter or membrane 833. In some aspects, the optional spring-loaded membrane or weighted bar can keep the removed clot confined to a specific region of the filter (e.g., within a transparent window) to provide an indication to the user regarding how much clot has been collected. For example, markers or hash marks 831 along the filter 824 edges as shown can estimate or provide an indication on the amount, size, or volume of clot removed. In other embodiments, weigh scales can be used in the clot collection cannister to indicate a weight of the clot removed. In some aspects, the weight can be converted to an estimated volume of the clot removed.

In another embodiment, referring to FIG. 8G, the filter 824 can use differential momentum of blood/clots flowing into the filter to separate or organize clots by size into various partitions 837. The momentum of larger or heavier clots will sort them into more distal partitions 837 relative to the inlet, and smaller clots will be sorted into the more proximal partitions. The arrangement can therefore automatically sort clots by size, volume, or weight into the filter during a thrombectomy procedure. As described above, the filter can be transparent or can have a transparent window to allow for visualization of the removed clots. Additionally, the filter 824 can include a membrane or filter 833 which allows blood to flow through the membrane to an outlet of the filter 824, while keeping the removed clots separated from the blood.

Operation of the vacuum source and cannister 804 also cause fluid, such as saline, to be pulled from the blood collection cannister 826 into the vacuum cannister 804. This causes separator 830 to expand or move within the blood collection cannister, pulling filtered blood/saline 827 into the blood collection cannister and/or into syringe(s) on the blood side of the cannister. FIG. 8B shows movement of the separator 830 indicated by the arrow through the blood collection cannister (in this embodiment, a plunger) resulting in blood 827 on a first side of the separator (e.g., the thrombus removal device side) and saline 829 on a second side of the separator (e.g., the vacuum source and cannister side). It can be seen how as the blood collection cannister fills with blood, the vacuum cannister fills with saline. In some embodiments, as shown in FIG. 8B, a blood return line 832 can optionally be connected to the patient to return blood 827 in the blood collection cannister back to the patient. A syringe can be added to the blood line to assist with blood return. This blood return line can be clamped off, for example, with pinch valves or clamps on either side of the syringe (not shown) to facilitate blood return to the patient when desired.

In some embodiments, referring to FIG. 8C, the blood collection cannister 826 can include more than one syringe, such as syringes 828a, 828b, 828c, etc. Adding more than syringe in series with the blood collection cannister can increase the volume of blood/clot that can be removed and/or returned from the patient before needing to empty the blood collection cannister and/or return blood to the patient. However, the volume of blood returned to the patient can still be monitored according to the number of syringes emptied during a procedure (e.g., 150-300 ml per syringe). As with the embodiment above, the syringes can be removably attached to the blood collection cannister.

FIG. 8D is a modification of the embodiment of FIG. 8B. The embodiment of FIG. 8D can include an additional fluid line 851 that fluidly couples the saline side of the blood collection cannister 826 to the saline source 806. Additionally, the system can include controllable valves 853a and 853b (e.g., any controllable three-way valve such as a three-way stopcock) at the connection between line 851 and the saline source 806 and at the junction between the blood collection cannister 826 and the blood return line. During a thrombectomy procedure, valve 853a can be controlled to create a flow of saline from the saline source 806 into the thrombectomy catheter, such as for jetting or irrigation of fluid.

Valve 853b can be controlled to allow blood removed from the patient to flow into the blood collection cannister 826 as controlled by the aspiration source 804 which causes separator 830 to move in the direction indicated by the arrow. After the thrombectomy procedure is completed, the valve 853a can be controlled to divert saline from saline source 806 into line 851 towards the saline side of the blood collection cannister 826 and valve 853b can be controlled to divert blood from the blood collection cannister into blood return line 832. In this blood return mode, the saline source fills the saline side of the blood collection cannister with saline, which drives separator 830 in the opposite direction of the arrow to push blood into the blood return line 832. The blood, which has already been filtered once with filter 824, can further be filtered with a second filter 855 before being returned to the patient. In some embodiments, blood return line 832 directs the (twice) filtered blood into the introducer sheath of the thrombectomy system for return to the patient. The embodiment of FIG. 8D can provide staged filtering with two different filter pore sizes (e.g., the first filter 824 can have a first (coarser) filter size and the second filter 855 can have a second (finer) filter size. During the thrombectomy procedure, aspiration of blood and clot into filter 824 can occur at a first pressure level. During blood return, blood can be filtered through filter 855 and returned to the patient at a second, higher pressure level, since the finer filter has a lower throughput.

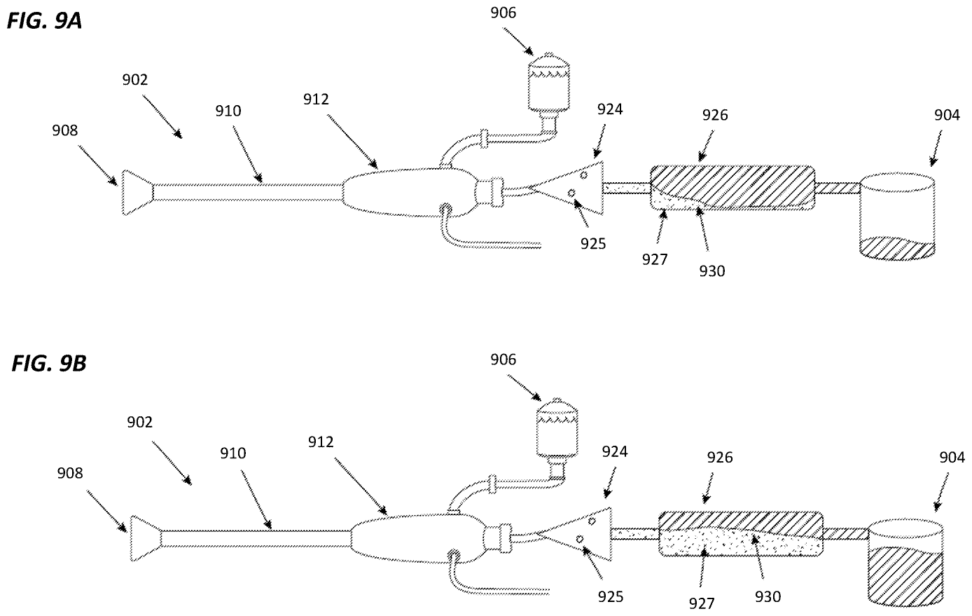

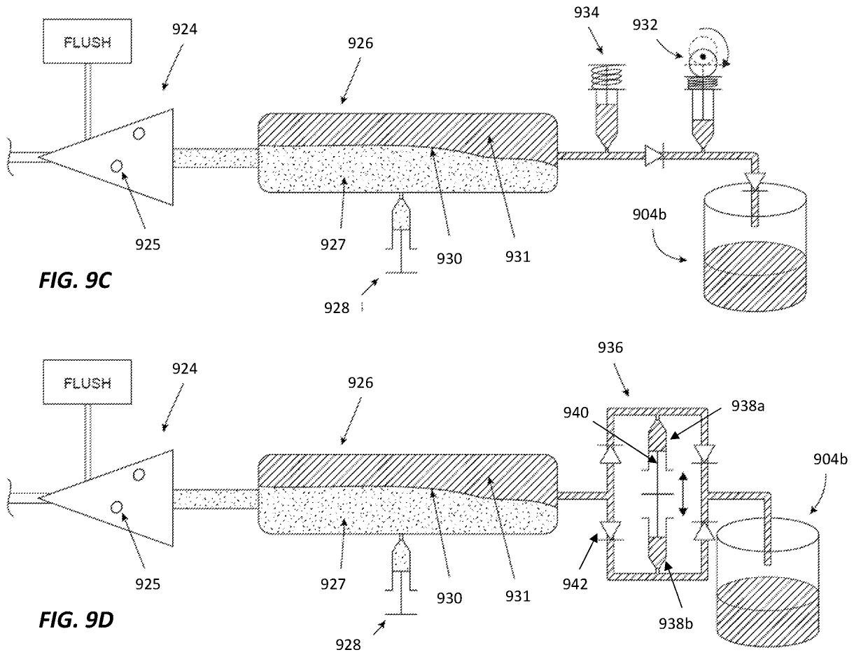

FIGS. 9A-9B show a similar embodiment to the thrombus removal system of FIGS. 8A-8B. However, in this embodiment, the blood collection cannister 926 includes a deformable diaphragm 930 instead of the plunger illustrated in FIGS. 8A-8B. However, the concept of operation is similar. The blood collection cannister 926 can be pre-filled with saline or another fluid. When the vacuum source and cannister 904 operates, fluid is pulled from the blood collection cannister 926 into the vacuum cannister 904, causing diaphragm 930 to move within the blood collection cannister as shown by the arrow. This results in blood/saline 927 removed from the patient being pulled into the blood collection canister on the opposite side of the diaphragm, as shown in FIG. 9B. The vacuum cannister 904 can fill with saline when saline in the blood collection cannister is displaced with blood/saline 927 removed from the patient. As with the embodiment described above in FIGS. 8A-8B, thrombus material or clots 925 can be collected on thrombus filter 924.

FIGS. 9C-9D show another embodiment of a thrombus removal system similar to the system described above in FIGS. 9A-9B. In FIG. 9C, the blood collection cannister 926 can include the previously described diaphragm 930. One or more syringes 928 can be disposed on the blood side of the blood collection cannister 926 to collect blood for return/redelivery to the patient. On the saline side of the blood collection cannister, a pump 932 such as a single piston pump can be configured to pull saline in from the saline side of the blood collection cannister and pump saline into the vacuum cannister 904b. One-way check valves positioned on either side of the pump 932 can prevent passage of saline back into the blood collection cannister. In other embodiments, the check valves can be actively driven valves to coincide with operation of the pump. In some examples, when the pump is driving or pushing saline into the vacuum cannister 904b, negative pressure on the inflow side can be maintained with an optional capacitive device such as the spring-loaded syringe or diaphragm 934. As the pressure decreases on the saline side, the plunger is pulled down against the spring.

FIG. 9D shows a similar embodiment to the one shown in FIG. 9C. Here, instead of a single piston pump as in the embodiment of FIG. 9C, this embodiment can include a pumping system 936 that includes two pumps or syringes 938a/938b with a common driveshaft or piston 940. In this embodiment, when pump 938a of the pumping system is pushing saline into the vacuum cannister 904b, pump 938b of the pumping system is pulling saline in from the saline side of the blood collection cannister. Valves 942, such as one-way check valves, or actively controlled valves, on the inlet and outlet sides of the top and bottom pumps can prevent saline from flowing back into the blood collection cannister. The spring-loaded syringe or diaphragm 934 of the FIG. 9C embodiment can optionally be employed in this embodiment as well as a way of controlling capacitance in the system.

In some embodiments, the thrombus filter 924 or 924 is removable from the system. After a thrombectomy procedure, the filter can be removed and strung out, extruded, or washed to separate removed clot/thrombus material from the filter. For example, a flush system can be connected to the filter, as shown in FIGS. 9C-9D, to flush the filter after or during a procedure to wash away blood for a better view of the collected clot materials. The removed clot/thrombus material can be measured, weighed, and/or saved for further diagnostics.

In additional embodiments, an electrical charge can be applied to the thrombus filter or to other aspects of the system, such as to the thrombus filter 824 of FIGS. 8A-8B or the thrombus filter 924 of FIGS. 9A-9D. Since blood is negatively charged, a positive charge can be applied to the thrombus filter to attract clot or thrombus material to the thrombus filter, while still allowing blood to pass through the pores/openings of the filter. For example, electrical leads can be attached to the filter and connected to an electrical source to apply the positive charge to the filter. In another embodiment, the polarity of the charge can be reversed, and a negative charge can be applied to the filter to help expel the collected thrombus material/clot after a procedure. In some embodiments, portions of the filter can be preferentially charged, thereby preferentially loading portions of the filter to prevent filter blockage. Additionally, captured clot can be segmented by, for example, the morphology of the clot (e.g., soft, medium, and hard versions). The segmentation can be implemented by tuning the charge applied and/or the positioning of elements (e.g., plates, prongs, etc.) in the blood collection cannister.

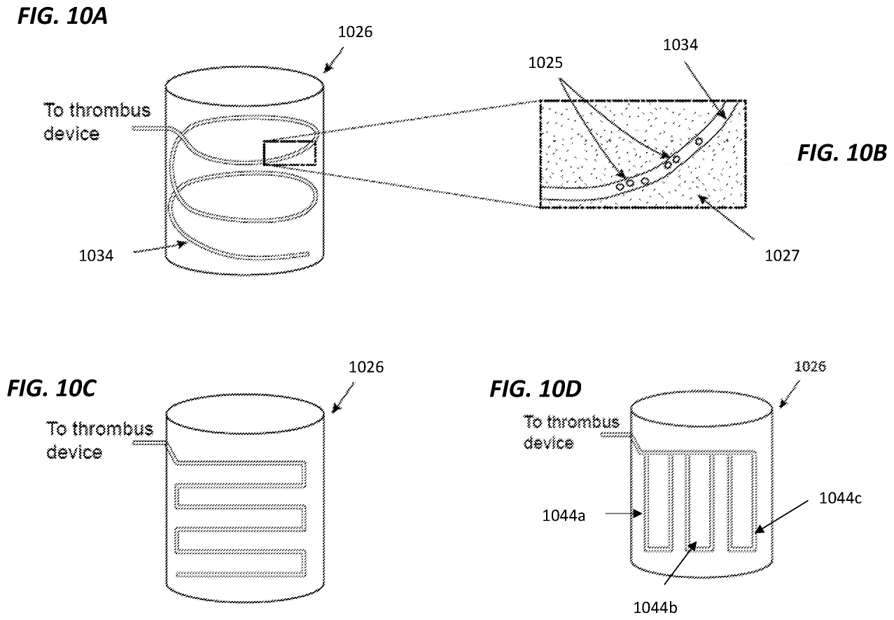

FIGS. 10A-10D illustrate alternate embodiments of a blood collection cannister 1026. In the FIG. 10A example, the blood collection cannister 1026 can include a sieve pathway 1034. The sieve pathway 1034 can comprise a spiral or tortuous pathway from the top to the bottom of the cannister, as shown in FIG. 10A. In another example, the sieve pathway 1034 can comprise a back and forth pathway as shown in FIG. 10C. FIG. 10B is a close-up view of the sieve pathway. The pathway can comprise a lumen, tube, channel, or the like with a plurality of openings or pores sized and configured to contain clots or thrombus material 1025 within the sieve pathway but allow blood/saline 1027 to fall or filter out of the pathway by way of gravity or an external vacuum source. As with the thrombus filter described above, the sieve pathway can include pore sizes on the order of 40 microns to allow blood but not clots or thrombus material to pass. The pathway can also include hashmarks or other units of measurement to provide an estimate of the amount of clot captured/removed within the pathway. In operation, vacuum applied to the blood collection cannister and the thrombus removal device will put blood/saline/clots out of the patient, through the thrombus removal device, and into the sieve pathway of the blood collection cannister. The filter or pore size of the sieve pathway allows blood to flow out of the sieve pathway and into the blood collection cannister, while containing clots or thrombus materials within the sieve pathway.

In some embodiments, an electrical charge can be applied to sieve pathway. Since blood is negatively charged, a positive charge can be applied to the sieve pathway to attract clot or thrombus material to the sieve pathway, while still allowing blood to pass through the pores/openings of the sieve pathway to collect in the blood collection cannister. For example, electrical leads can be attached to the sieve pathway and connected to an electrical source to apply the positive charge to the sieve pathway. In another embodiment, the polarity of the charge can be reversed, and a negative charge can be applied to the sieve pathway to help expel the collected thrombus material/clot after a procedure.

FIG. 10D is another embodiment of a blood collection cannister 1026 that can include a plurality of electrically charged prongs 1044a-1044c configured to capture clot and segment the clot by, for example, the morphology (e.g., soft, medium, hard, etc.) of the clot. The prongs can be individually tuned with a desired or chosen electrical charge to attract the desired clot morphology. For example, the first prong 1044a may be charged with a first charge configured to attract a first type of clot or clot morphology, the second prong 1044b may be charged with a second charge configured to attract a second type of clot or clot morphology, and the third prong 1044c may be charged with a third charge configured to attract a first type of clot or clot morphology. In some embodiments, one or more prongs may be tuned with the same or similar charge if the majority of clot being removed is attracted to that specific charge tune. While the cannister of FIG. 10D shows three prongs, it should be understood that the cannister can include fewer or more prongs depending on the application.

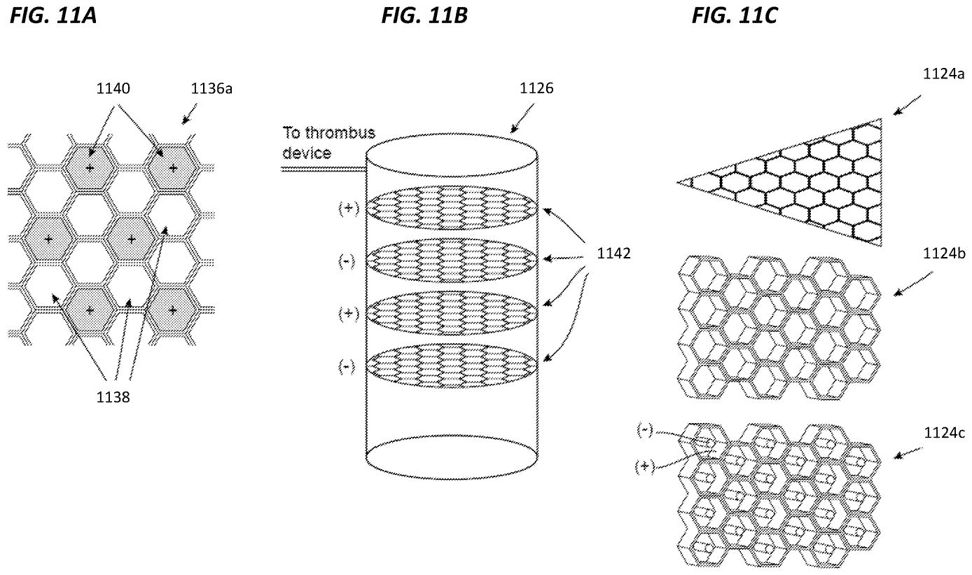

FIGS. 11A-11C illustrate another embodiment of a structure that can be incorporated into thrombus filters and/or blood collection cannisters of a thrombus removal system. In FIG. 11A, a honeycomb structure 1136 is shown that can include a plurality of openings 1138 (illustrated in white) interspersed with closed sections 1140 (illustrated in grey shading). The openings can have pore sizes configured to allow the passage of blood/fluid but not clot or thrombus material, as described above. In some embodiments, the closed sections 1140 can include a positive charge indicated by (+) to attract clot or thrombus material while still allowing blood and/or fluid removed from the patient to pass through the openings 1138.

In FIG. 11B, one or more layers 1142 of the honeycomb structure can be implemented within a blood collection cannister 1126, as shown. Gravity or vacuum can pull blood, saline, and/or removed clot/thrombus material into the blood collection cannister 1126, which can filter down through the layers 1142. Clot or thrombus material can collect on the closed sections of each layer, while blood can flow down through the openings to collect within the cannister. As with the embodiment of FIG. 11A, the layers can include a positive charge, particularly on the closed sections, to further attract clot or thrombus material. While the illustrated embodiment illustrates alternating positive and negative charges, it should be understood that the various layers 1142 can be individually controlled with the desired charge (e.g., all positive charge or all negative charge, or some combination thereof). Additionally, the layers 1142 can be individually tuned to attract or sort different types or morphologies of clot. For example, the charge level of a first layer may be tuned to attract or sort soft clot, a second layer tuned to attract or sort medium clot, a third layer tuned to attract or sort hard clot, etc. A syringe can optionally be coupled to the bottom of the cannister for collection of blood and blood return to the patient. Optionally, the cannister 1126 can be coupled to another blood collection cannister (not shown) to allow all the blood to be separately collected.

In FIG. 11C, the same concept can be applied to a thrombus filter 1124a, 1124b, or 1124c, which can be the thrombus filter from the embodiment of FIGS. 8A-8B and 9A-9B. In this example, the thrombus filter can include closed sections (optionally charged) designed and configured to collect removed/bind thrombus material while the openings allow blood and other fluids to pass through the filter (such as into the blood collection cannister previously described). The filter 1124b can include a honeycomb structure with a plurality of open lumens, the surface of which can be charged. When the filter is positively charged it has a greater affinity for clots, therefore it allows blood to flow through the lumens while preventing clot from passing by capturing it on the surface. Filter 1124c can further include rods in the lumens. The rods can be charged to a negative charge (−) and the surface of the honeycomb structure lumens can be positively charged (+) to cause clot to attract to the honeycomb structures. The width or length of these lumens can be increased to provide a longer pathway for blood and clot to flow through, thereby increasing the surface area available for the clot to be collected.

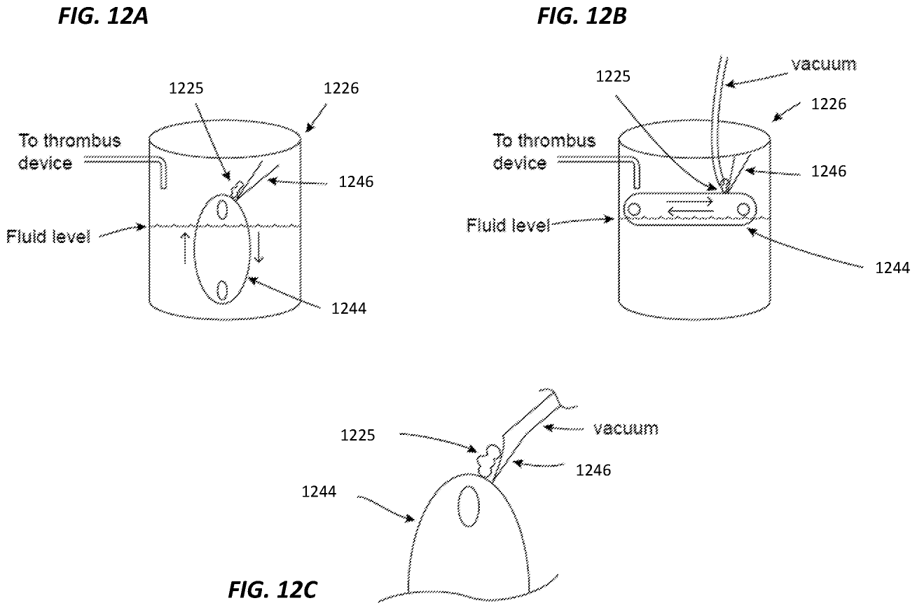

FIGS. 12A-12C illustrate another embodiment of a blood collection cannister 1226. The blood collection cannister 1226 can replace any of the other blood collection cannisters described herein. The blood collection cannister 1226 can include a conveyor belt 1244 with two or more pulleys and a scraper 1246. The conveyor belt 1244 can be positively charged. FIG. 12A shows an embodiment in which the conveyor belt is vertically arranged within the blood collection cannister, and FIG. 12B shows an embodiment in which the conveyor belt is horizontally arranged within the blood collection cannister. When blood, fluid, and thrombus material are removed from a patient into the blood collection cannister 1226, the positively charged conveyor belt can operate to attract thrombus material from below the fluid level to be pulled onto the conveyor belt. A scraper 1246 (FIGS. 12A and 12B)) can contact or nearly contact the conveyor belt to grab or remove the thrombus material from the conveyor belt. In some embodiments, the conveyor belt can be selectively controlled to operate only at a specified time, such as after a procedure is complete. The conveyor belt and/or scraper can be lifted out of the cannister to remove the separated thrombus material from the blood collection cannister, resulting in only blood/saline remaining in the cannister.

It should be understood that the honeycomb structure of FIG. 12A could also be applied to the sieve filter of FIGS. 11A-11B.

In an alternative embodiment, as shown in FIG. 12C, the scraper can be replaced with a vacuum nozzle to directly remove the separated thrombus material from the conveyor belt. In some examples, this aspirated thrombus material can be stored in a separate thrombus cannister. FIG. 12B shows an embodiment with both a scraper and a vacuum nozzle.

In some embodiments, the positive charge on the conveyor belt can be turned off or negated near the scraper/vacuum nozzle. In another embodiment, the scraper and/or vacuum nozzle can have a negative charge to negate the positive charge on the conveyor belt, for easier removal/scraping of the removed thrombus material from the conveyor belt. In some examples, alternating a negative and positive charge can cause the clot to dissolve. This disclosure typically wants to avoid dissolving the clot until potentially after a procedure and after the clinician has seen the amount of clot removed.

While the embodiments herein have been described as being intended to remove thrombi from a patient's vasculature, other applications of this technology are provided. For example, the devices described herein can be used for breaking up and removing hardened stool from the digestive tract of a patient, such as from the intestines or colon of a patient. In one embodiment, the device can be inserted into a colon or intestine of the patient (such as through the anus) and advanced to the site of hardened stool. Next, the aspiration system can be activated to engage the hardened stool with an engagement member (e.g., funnel) of the device. Finally, the jets or irrigation can be activated to break off pieces of the hardened stool and aspirate them into the system. Any of the techniques described above with respect to controlling the system or removing clots can be applied to the removal of hardened stool.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology. Accordingly, the systems described herein are not limited to those configurations expressly identified, but rather encompasses variations and alterations of the described systems.

### Conclusion

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A thrombus removal system, comprising:
an elongate shaft;
an aspiration lumen extending along the elongate shaft;
a vacuum source fluidly coupled to the aspiration lumen;
a thrombus filter disposed along the aspiration lumen; and
a blood collection cannister disposed proximally from the thrombus filter along the aspiration lumen, the blood collection cannister including a moveable separator that divides the blood collection cannister into first and second chambers, wherein operation of the vacuum source causes clot removed from the patient to collect on the thrombus filter and blood removed from the patient to collect in the first chamber.

2. The system of claim 1, wherein operation of the vacuum source further causes saline to flow into the second chamber and a vacuum cannister.

3. The system of claim 1, wherein the filter has a pore size of up to 40 microns.

4. The system of claim 1, wherein the thrombus filter is configured to allow blood to pass but not allow thrombus to pass.

5. The system of claim 1, wherein the thrombus filter is positively charged.

6. The system of claim 1, wherein the separator comprises a plunger.

7. The system of claim 1, wherein the separator comprises a diaphragm.

8. The system of claim 1, further comprising at least one saline source fluidly coupled to the second chamber.

9. The system of claim 8, wherein the at least one saline source is removable from the blood collection cannister.

10. The system of claim 1, further comprising a blood return line fluidly coupled to the first chamber.

11. The system of claim 1, wherein the thrombus filter comprises a honeycomb structure.

12. The system of claim 1, wherein the honeycomb structure comprises a plurality of openings interspersed between closed sections.

13. The system of claim 12, further comprising an electrical system configured to apply a positive charge to the closed sections.

14-46. (canceled)
